(19) United States
(12) Patent Application Publication
(10) Pub. No.: US 2019/0240367 A1
(43) Pub. Date: Aug. 8, 2019

(54) SPILL-PROOF, PASSIVE AIR FRESHENER REFILL CARTRIDGE

(71) Applicant: New Market Concepts, LLC (a Nevada LLC), Chicago, IL (US)

(72) Inventors: Rami Sidawi, Milton (CA); Asad Rahman, Keswick (CA); Jeremiah David, Chicago, IL (US)

(73) Assignee: New Market Concepts, LLC (a Nevada LLC), Chicago, IL (US)

(21) Appl. No.: 15/890,332

(22) Filed: Feb. 6, 2018

(51) Int. Cl. A61L 9/12

(52) U.S. Cl. CPC A61L 9/127; A61L 2209/133

(57) ABSTRACT

A spill-proof, passive air freshener refill cartridge, effective for at least sixty days, includes both a wick assembly and a supply canister filled with a volatile liquid fragrance. The volatile liquid fragrance is dispersed into the air by a caged tripartite wick assembly. The tripartite wick assembly has a cylindrical wall component, from which most dispersal occurs, a circular laminar component, and a pointed vertical pole component. The circular laminar component is in contact with both the base of the cylindrical wall component and the top of the vertical pole component, which extends downwardly from the center of the circular laminar component into the supply canister containing the volatile liquid fragrance. The volatile liquid fragrance is transported via the pointed vertical pole component to the circular laminar component to the cylindrical wall component.

### FIELD OF THE INVENTION

This invention relates, generally, to fragrance dispensers and, more particularly, to air freshener refills which employ evaporation as a means to dispense a volatile liquid fragrance compound.

### BACKGROUND OF THE INVENTION

The annual market for air freshener products in the U.S. is over $3 billion. Plug-in type air freshener products account for a third of that market. Over the past decade, battery-operated, metered aerosol fragrance dispensers have become popular in commercial settings such as public and office restrooms. As these devices are typically somewhat complex, in that they incorporate a DC electric motor, a gear drive operated by the motor which actuates the spray valve, an aerosol fragrance canister, and a programmable timer for adjusting the time interval between sprays, average retail prices of around $50 were too costly to generate widespread demand for home use. However, prices have dropped dramatically. For example, Wal-Mart now sells a programmable Air Wick® dispenser with one aerosol fragrance canister for less than $10. As the cost of a replacement fragrance canister is nearly $4, it appears that American Home Products Corporation may be selling the Air Wick® fragrance dispenser at less than cost, a marketing strategy learned from Gillette's sale of razor blades and Hewlett-Packard's sale of printer ink cartridges.

One problem associated with the motor-operated fragrance dispensers is that the moving mechanical parts are the components most likely to fail over time. A simpler, more durable solution employs a micro-perforated plate secured to a piezoelectric vibrator that is energized by an AC voltage. Such devices have been used as humidifiers and as aerosol fragrance dispensers. U.S. Pat. No. 5,297,734 to Kohji Toda discloses such an ultrasonic vibrating device. The micro-perforations are conical and larger in diameter on the supply side, and the piezoelectric vibrator operates at a frequency of about 100 kHz.

U.S. Pat. No. 5,657,926, also to Kohji Toda, discloses an ultrasonic atomizing device that employs electrical feedback from the piezoelectric vibrator to generate a resonant frequency of greater amplitude to vibrate the micro-perforated plate.

U.S. Pat. No. 6,293,474 to Thomas A. Helf, et al. discloses a wick-based delivery system to provide liquid to a micro-perforated plate that is vibrated by a piezoelectric device.

U.S. Pat. No. 6,341,732 to Frederick H. Martin, et al. discloses a method and apparatus for maintaining control of liquid flow in a vibratory atomizing device. As with the '474 Patent to Helf, et al., liquid if provided to a vibrating orifice plate by a wick. However, liquid that is not expelled by the vibrating plate drains back through the plate and absorbed by the wick, which then provides it again to the vibrating plate.

Simpler still are the air freshener dispensers in which either a volatile liquid fragrance evaporates or a solid fragrance block sublimates and the vaporized fragrance compound is dispersed into the air. Vaporization or sublimation and dispersal of the fragrance compound can be accelerated by providing a fan that blows air across the fragrance supply.

### SUMMARY OF THE INVENTION

The present invention provides a passive air freshener refill cartridge, which is effective for at least 60 days, that includes both a wick assembly and a supply canister filled with a volatile liquid fragrance. The volatile liquid fragrance is dispersed into the air by a caged tripartite wick assembly. The tripartite wick assembly has a cylindrical wall component, from which dispersal occurs, a circular laminar component, and a pointed vertical pole component. The circular laminar component is in contact with both the base of the cylindrical wall component and the top of the vertical pole component, which extends downwardly from the center of the circular laminar component into the supply canister containing the volatile liquid fragrance. The volatile liquid fragrance is transported via the pointed vertical pole component to the circular laminar component to the cylindrical wall component. Although for the preferred embodiment of the invention, all wick components are made of open-pore polyethylene foam, other wick materials, such as fabrics, can be used with success. The air freshener refill is designed as a replacement for air freshener cartridges such as those that are installed in Rubbermaid® T-Cell and Kimberly-Clark KimCare® air freshener dispenser products. The refill cartridge is manufactured with the liquid fragrance supply capped with a foil and polyethylene membrane. That membrane can be pierced by peeling away a circumferential strip on the refill cartridge, which will then enable the caged tripartite wick assembly to be pressed in a downward direction, so that the pointed vertical pole component pierces the membrane and a major portion of the pole portion enters the mouth of the liquid fragrance supply canister so that the liquid fragrance can flow, through capillary action, up the vertical pole component, into the circular laminar component and, finally, into the cylindrical wall component of the tripartite wick. The air freshener refill cartridge is spill proof on account of the vertical pole component of the tripartite wick engaging the mouth of the liquid fragrance supply canister in a press fit, and there being only one route for fragrance to exit the supply canister, and that is through the wick.

### BRIEF DESCRIPTION OF THE DRAWINGS

FIG. 1 is an isometric view of the reservoir of the fragrance supply canister;

FIG. 2 is a top plan view of the reservoir of the fragrance supply canister;

FIG. 3 is bottom plan view of the reservoir of the fragrance supply canister;

FIG. 4 is side elevational view of the reservoir of the fragrance supply canister;

FIG. 5 is a cross-sectional view of the reservoir of the fragrance supply canister, taken through section plane 5-5 of FIG. 4;

FIG. 6 is an isometric view of a first embodiment top cover of the fragrance supply canister;

FIG. 7 is a top plan view of the first embodiment top cover of the fragrance supply canister;

FIG. 8 is a side elevational view of the first embodiment top cover of the fragrance supply canister;

FIG. 9 is a cross-sectional view of the first embodiment top cover of the fragrance supply canister, taken through section plane 9-9 of FIG. 8;

FIG. 10 is s closeup view of the circular portion 10 of FIG. 9, showing the thread which secures the first embodiment top cover to the bottom portion;

FIG. 11 is an isometric view of the O-ring that seals the joint between the upper and lower portions of the fragrance supply canister;

FIG. 12 is a top plan view of the O-ring of FIG. 14;

FIG. 13 is a side elevational view of the O-ring of FIG. 14;

FIG. 14 is a cross-sectional view of the O-ring of FIG. 14, taken through section plan 14-14 of FIG. 13;

FIG. 15 is an isometric view of the wick container;

FIG. 16 is a top plan view of the wick container;

FIG. 17 is a bottom plan view of the wick container;

FIG. 18 is a side elevational view of the wick container;

FIG. 19 is a cross-sectional view of the wick container, taken through section plane 19-19 of FIG. 18;

FIG. 20 is an isometric view of the cylindrical wall component of the tripartite wick;

FIG. 21 is side elevational view of the cylindrical wall component of the tripartite wick;

FIG. 22 is a bottom plan view of the cylindrical wall component of the tripartite wick;

FIG. 23 is an isometric view of a first embodiment of the circular laminar component and a vertical pole component of the tripartite wick, which are combined as a unitary structure;

FIG. 24 is a side elevational view of the first embodiment of the unitized circular laminar component and a vertical pole component of the tripartite wick;

FIG. 25 is a top plan view of the first embodiment of the unitized circular laminar component and a vertical pole component of the tripartite wick;

FIG. 26 is an isometric view of a second embodiment of the circular laminar component and a vertical pole component of the tripartite wick, which are combined as a unitary structure;

FIG. 27 is a side elevational view of the second embodiment of the unitized circular laminar component and a vertical pole component of the tripartite wick;

FIG. 28 is a top plan view of the second embodiment of the unitized circular laminar component and a vertical pole component of the tripartite wick;

FIG. 29 is an isometric view of a four-point blade that fits over the blunt bottom end of the second embodiment of the unitized circular laminar component and the vertical pole component of the tripartite wick;

FIG. 30 is a bottom plan view of the four-point blade of FIG. 29;

FIG. 31 is a side elevational view of the four-point blade of FIG. 29;

FIG. 32 is a cross-sectional view of the four-point blade of FIG. 29, taken through section plane 32-32 of FIG. 31;

FIG. 33 is a side elevational view of a second embodiment top cover of the fragrance supply canister;

FIG. 34 is a top plan view of the screw-on cap;

FIG. 35 is an isometric view of the screw-on cap of FIG. 34;

FIG. 36 is a bottom plan view of the screw-on cap of FIG. 34;

FIG. 37 is a side elevational view of the screw-on cap of FIG. 34;

FIG. 38 is a cross-sectional view of the screw-on cap of FIG. 34, taken through section plane 38-38 of FIG. 37;

FIG. 39 is a closeup view of that portion of the securing thread that is encircled in FIG. 38;

FIG. 40 is an exploded view of the air freshener cartridge having a second embodiment wick pole component assembly;

FIG. 41 is an exploded view of the air freshener cartridge having a first embodiment wick pole component assembly;

FIG. 42 is an isometric view of the assembled air freshener cartridge assembly with the protector strip still in place and the seal on the opening of the liquid fragrance supply canister unpunctured;

FIG. 43 is a side elevational view of the assembled air freshener cartridge assembly with the protector strip still in place and the seal on the opening of the liquid fragrance supply canister unpunctured;

FIG. 44 is a top plan view of the assembled air freshener cartridge assembly with the tear-away strip still in place and the seal on the opening of the liquid fragrance supply canister top cover unpunctured;

FIG. 45 is a side elevational view of the assembled air freshener cartridge assembly with the protector strip removed, and the caged wick assembly pressed downward to puncture the seal on the opening of the liquid fragrance supply canister and the wick inserted into the liquid within the canister;

FIG. 46 is a top plan view of the assembled air freshener cartridge assembly with the protector strip removed;

FIG. 47 is a cross-sectional view of FIG. 43, taken through section line 47-47.

FIG. 48 is a cross-sectional view of the air freshener cartridge of FIG. 43, after the tear-away strip has been removed and the tripartite wick and wick cage have been downwardly pushed;

FIG. 49 is a cross-sectional view of FIG. 44, taken through section line 49-49;

FIG. 50 is a cross-sectional view of the air freshener cartridge of FIG. 44, after the tear-away strip has been removed and the tripartite wick and wick cage have been downwardly pushed;

FIG. 51 is a front elevational view of a passive air freshener dispenser;

FIG. 52 is a front elevational view of the passive air freshener dispenser of FIG. 51, with the front cover removed;

FIG. 53 is a front elevational view of the passive air freshener dispenser of FIG. 51, with the front cover removed and the air freshener cartridge of FIG. 43 installed therein;

FIG. 54 is a front elevational view of a fan-equipped air freshener dispenser;

FIG. 55 is a front elevational view of the fan-equipped air freshener dispenser of FIG. 54, with the front cover removed; and

FIG. 56 is a front elevational view of the fan-equipped air freshener dispenser of FIG. 54, with the front cover removed and the air freshener cartridge of FIG. 43 installed therein.

### PREFERRED EMBODIMENT OF THE INVENTION

The various embodiments of the new air freshener cartridge will now be described in detail with reference to the attached drawing figures. It is to be understood that the drawings are not necessarily drawn to scale and that they are intended to be merely illustrative of the invention.

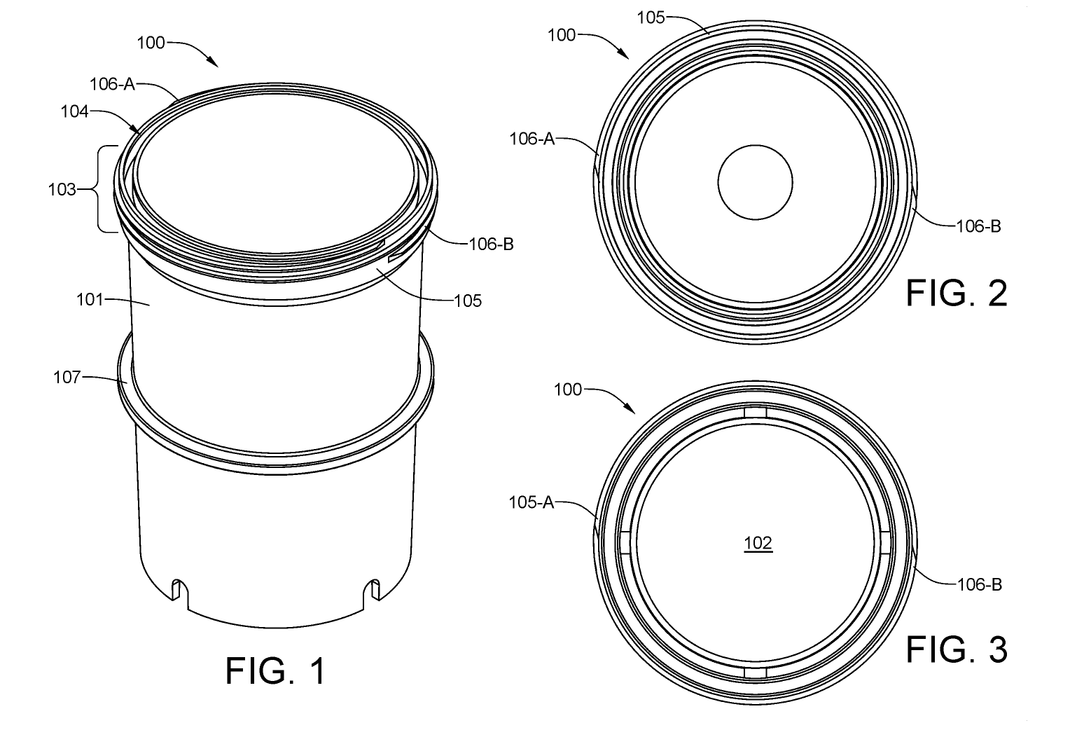

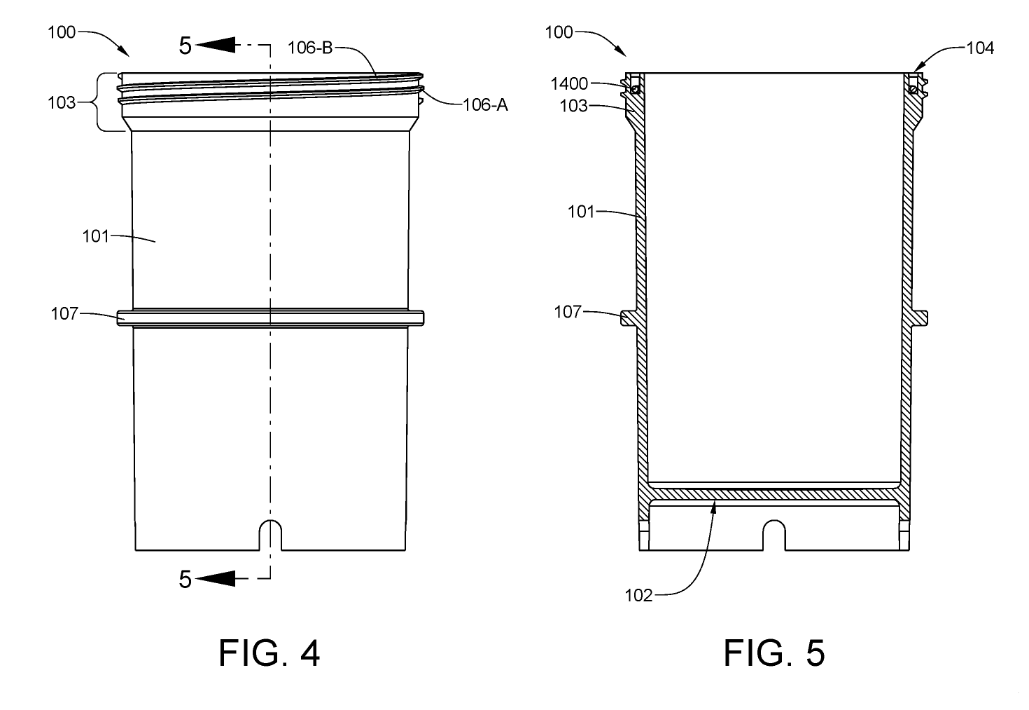

Referring now to FIGS. 1, 2, 3, 4 and 5, the reservoir 100 of the fragrance supply canister is cup shaped with an open mouth, and has a stepped, nearly cylindrical wall 101 and a generally planar floor 102 which is integral with the cylindrical wall 101. The wall 101 is slightly tapered so that it can be molded and subsequently removed from the mold. An uppermost portion 103 of the reservoir 100 is thickened, having an upward-facing annular groove 104 in the top edge 105 of the thickened portion 103. The exterior surface of the thickened portion 103 is equipped with a pair of parallel external right-hand threads 106-A and 106-B. Referring now, specifically, to FIG. 5, an O-ring 1400 can be seen that seals the joint between the top cover 600 and the reservoir 100. The O-ring 1400 will be shown in greater detail in FIGS. 14 to 17. A lock-out flange on the cylindrical wall 101 prevents, or at least hampers, installation of the air freshener cartridge in an unauthorized air freshener dispenser.

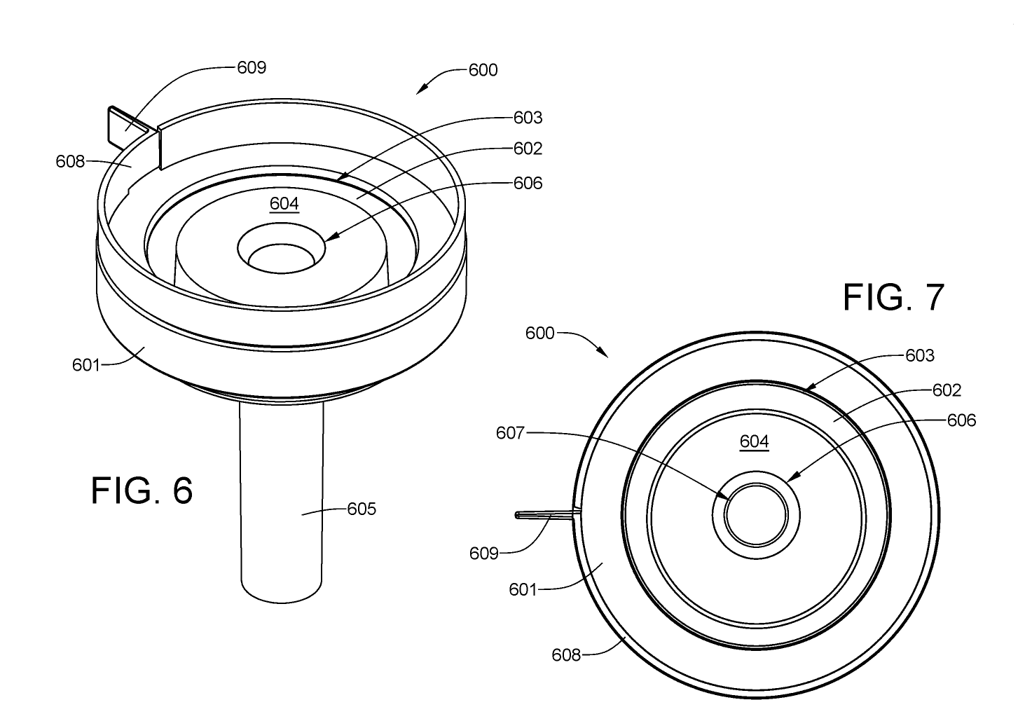

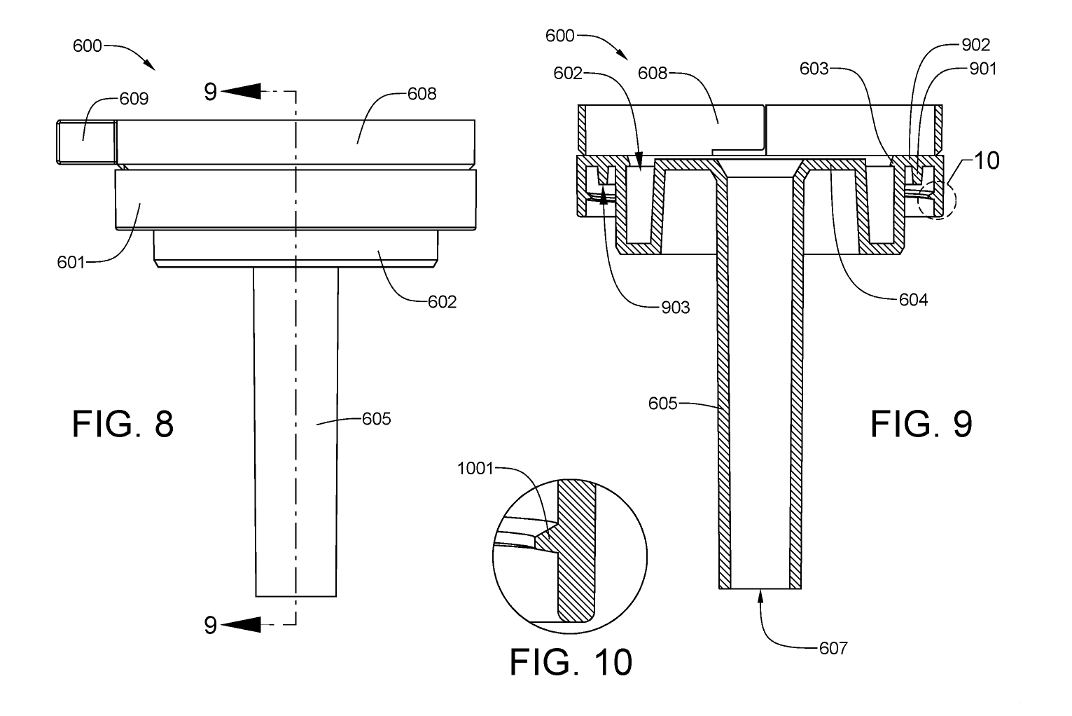

Referring now to FIGS. 6, 7, 8, 9 and 10, a first embodiment top cover 600 of the fragrance supply canister has an inverted circumferential channel 601 with an internal thread 1001 (see FIG. 10) within the inverted channel 601 that engages the external right-hand threads 106-A and 106-B of the bottom portion 100 of the fragrance supply canister. The top cover 600 also has a wick holder installation trench 602 that is unitary with the inverted channel 601, and which has an annular barb 603 near the top thereof that captures the lower cylindrical extension 1805 of the wick holder 1800 in a first position for shipping and pre-use storage and in a second, in-use, position. A central lid portion 604 is integral with both the installation trench 602 and a vertically-oriented tubular well 605 that has a tapered upper opening 606 in the center of the central lid portion 604 and a lower opening 607 that is covered with a circular foil and polyethylene membrane (see FIGS. 43, 44, 50 and 51). The first embodiment top cover 600 has a tear-away circumferential strip 608 with a pull tab 609 that, when removed, enables the tripartite wick to be pushed in a downward direction, causing the pole portion of the wick to pierce the foil and polyethylene membrane and enter the supply of liquid fragrance contained in the fragrance supply canister. Finally, the top cover 600 has a downward-projecting annular flange 901, that is integral with the ceiling 902 of the inverted channel 601, and that fits into the annular groove 104. A lower surface of the annular flange 901 contacts and compresses the O-ring 1400.

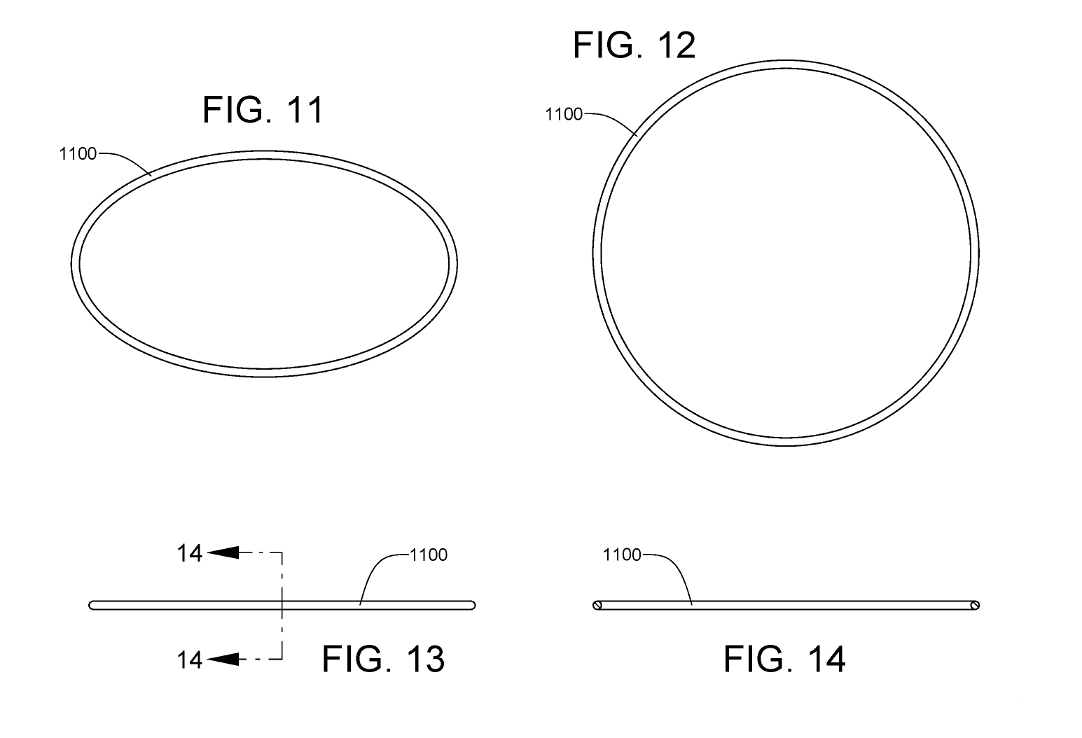

Referring now to FIGS. 11, 12, 13 and 14, an O-ring 1100 is shown that seals the joint between the bottom portion 100 and top cover 600 of the fragrance supply canister. The O-ring 1100 is compressed between the lower surface 903 of the annular flange 901 and the bottom of the upward facing annular groove 104 that is at the top of reservoir 100.

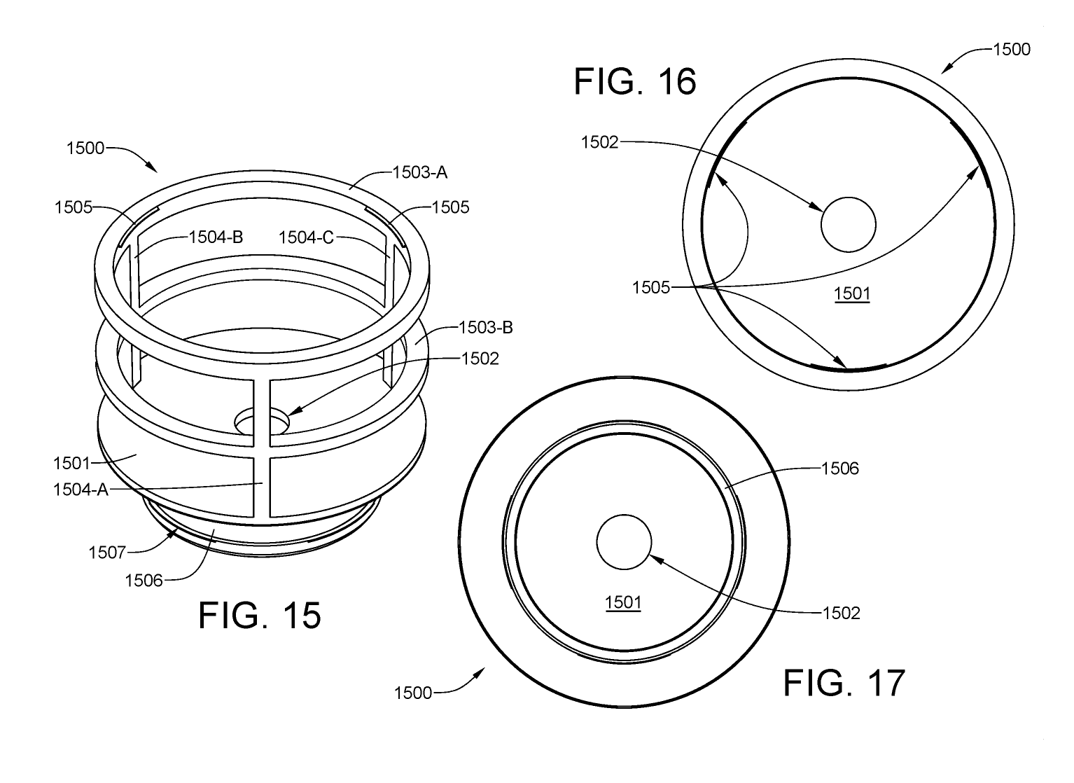

Referring now to FIGS. 15, 16, 17, 18 and 19, the wick holder 1500 is a unitary structure formed from a base 1501, which has a central aperture 1802, and a cage structure that is made up of two annular rings (1503-A and 1503-B) and three equiangularly-spaced vertical posts (1504-A, 1504-B and 1504-C), which interconnect the base 1501 and the two annular rings 1503-A and 1503-B. Three equiangularly-spaced barbs 1505 (only two are visible in FIG. 15; all three are visible in FIG. 16) trap the cylindrical wall component 2000 of the wick. The base also has a lower cylindrical extension 1506 that clips into the wick holder installation trench 602 of the top cover 600. The cylindrical extension 1506 has an annular shelf 1507 that is captured by the annular barb 603 of the top cover 600 in the first shipping and storage position.

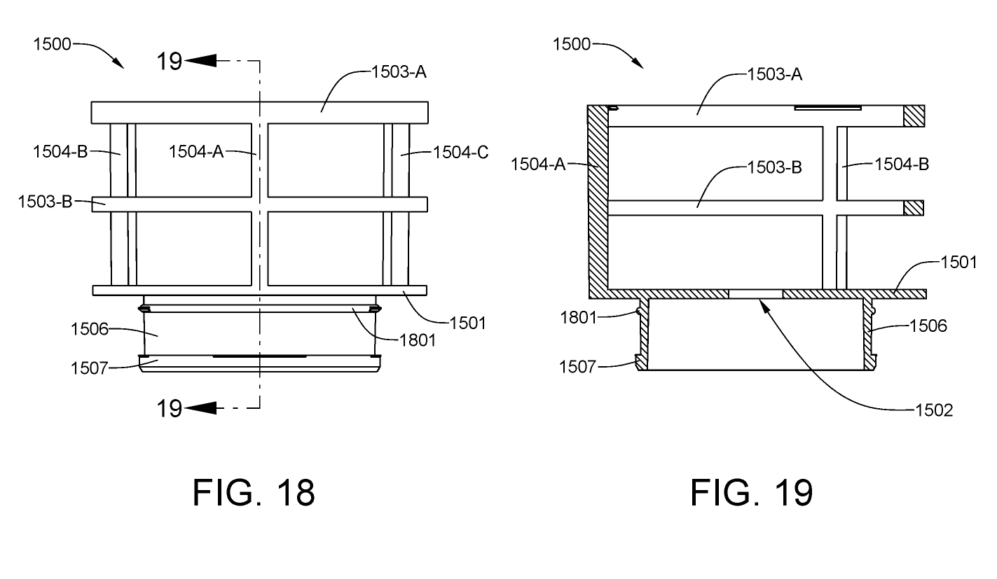

Referring now, specifically, to FIGS. 18 and 19, an annular lip 1801 is captured by the annular barb 603 in the second, in-use, position after the wick holder 1500 and tripartite wick are pushed down to rupture the foil and polyethylene seal 4601 and immerse the bottom end of the vertical pole component of the wick (2300-B or 2600-B) in the liquid fragrance.

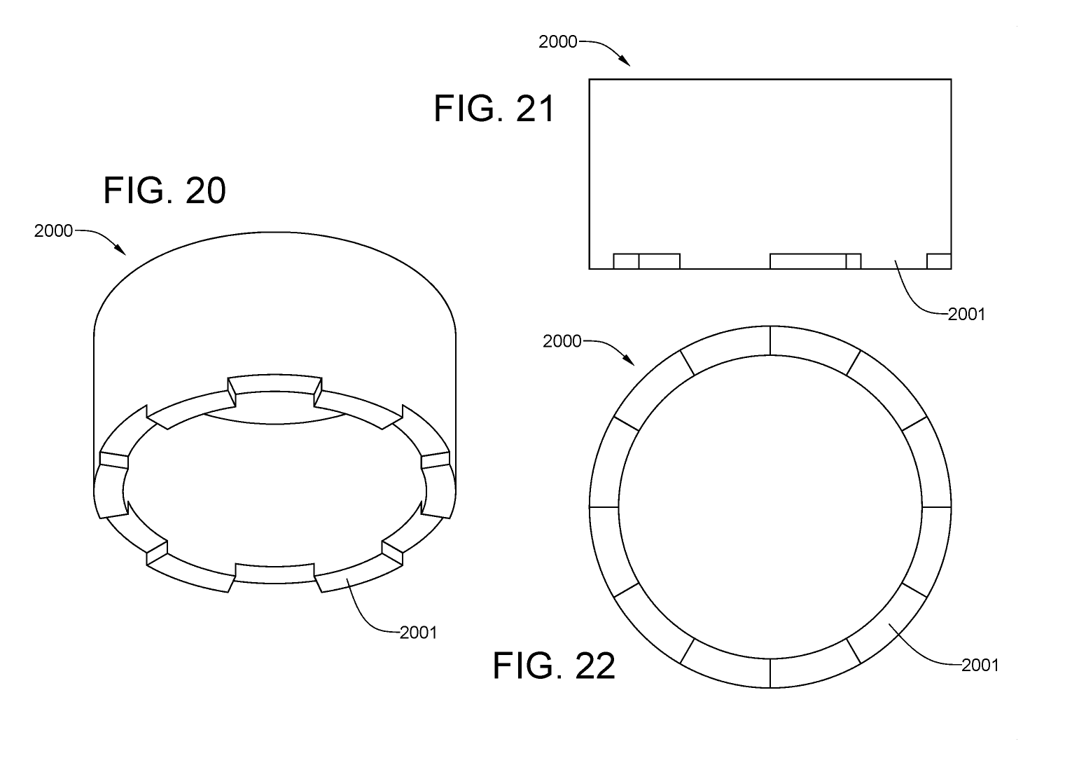

Referring now to FIGS. 20, 21 and 22, the cylindrical wall component 2000 of the tripartite wick, which is castellated along its lower edge 2001, fits entirely within the wick container 1500. It, like the other two components of the wick, is made of sintered open-pore polyethylene foam. The polyethylene material is initially in powdered or granular form. It is then compressed and heated in a mold to form the desired shape. The remarkable characteristic of sintered polyethylene is its micro porosity. As polyethylene is hydrophobic, liquid fragrances which are dissolved in a non-polar solvent are required.

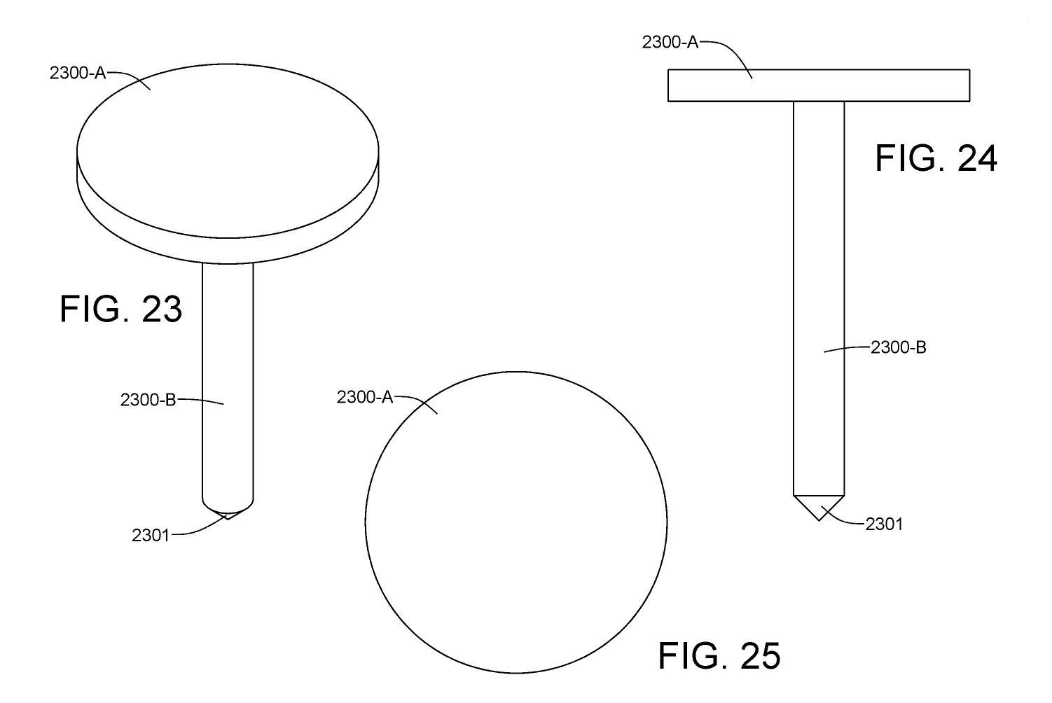

Referring now to FIGS. 23, 24 and 25, a first embodiment circular laminar disc component 2300-A and a vertical pole component 2300-B of the tripartite wick, which are combined as a unitary structure, has a sharpened conical point 2301 at the lower end of the vertical pole component 2300-B. The central axis of the vertical pole component 2300-B is perpendicular to the laminar component 2300-A. When the wick container 1500 and tripartite wick (2000, 2300-A and 2300-B) are forced in a downward direction after tearing away the tear-away strip 706 from the top cover 600 of the liquid fragrance canister, the sharpened point 2301 pierces the foil and polyethylene membrane so that the vertical pole component 2300-B can enter the interior of the fragrance canister and be immersed in the liquid fragrance.

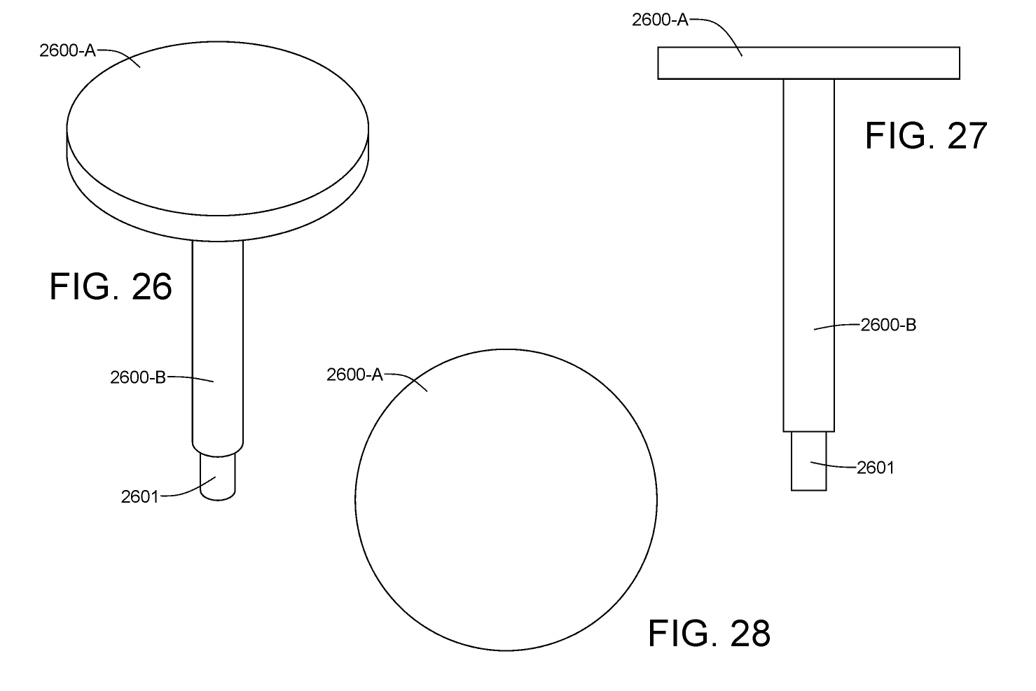

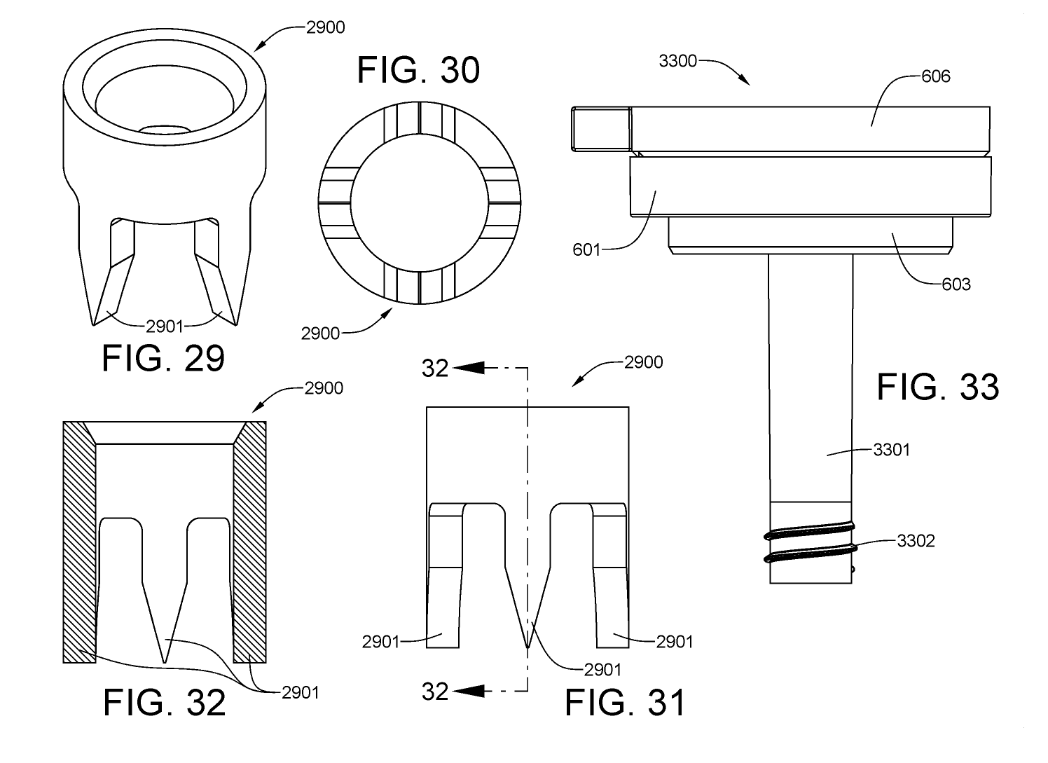

Referring now to FIGS. 26, 27 and 28, a second embodiment circular laminar component 2600-A and a vertical pole component 2600-B are also combined as a unitary structure. Instead of having the sharpened point 2301 of the first embodiment, the lower end 2601 of pole component 2600-B is reduced in diameter so that it can receive a four-pointed blade (see FIGS. 29, 30, 31 and 32).

Once the vertical pole component 2300-B or 2600-B, respectively, of either the first or second embodiment of the circular laminar component/vertical pole component is immersed in the liquid fragrance, the liquid fragrance climbs the pole component via capillary action, enters the laminar disc component and is subsequently transferred to the cylindrical wall component 2000, from whence it evaporates into the atmosphere at a controlled rate that is dependent on both ambient temperature and the rate of air flow over the wick components which are exposed to the atmosphere.

The cylindrical wall component 2000 and the circular laminar component 2300-A and 2600-A, respectively, of both the first and second embodiments of the circular laminar/vertical pole component fit within the wick container 1500. Except where covered by the cage structure, the cylindrical wall component 2000 is fully exposed to the atmosphere. The top of the circular laminar component (both 2300-A and 2600-A), except for the outer edges thereof which contact portions of the castellated edge 2001 is also exposed to the atmosphere. The pole component of both the first and second embodiments of the circular laminar/vertical pole component projects through the central aperture 1501 in the base 1501 of the wick container 1500.

Referring now to FIGS. 29, 30, 31 and 32, a four-pointed blade 2900 is designed to fit over the lower end of pole component 2600-B. Its outer diameter is less than the inside diameter of the cylindrical well 704 so that it can pass through the opening 705. It should also be emphasized that the vertical pole components 2300-B and 2600-B of the first and second embodiments of the unitary circular laminar and vertical component has an outside diameter that is slightly larger than the inside diameter of the cylindrical well 704, so that the vertical pole components are slightly compressed to prevent spills of the liquid fragrance if the in case the air freshener cartridge is tipped over.

Referring now to FIG. 33, a second embodiment top cover 3300 of the fragrance supply canister is identical to the first embodiment top cover 600 with the exception that the bottom portion of the vertically-oriented generally cylindrical well 3301 is equipped with an external thread 3302. Like the first embodiment top cover 600, the second embodiment top cover 3300 also has an opening 3303 at the bottom end of the cylindrical well 3301.

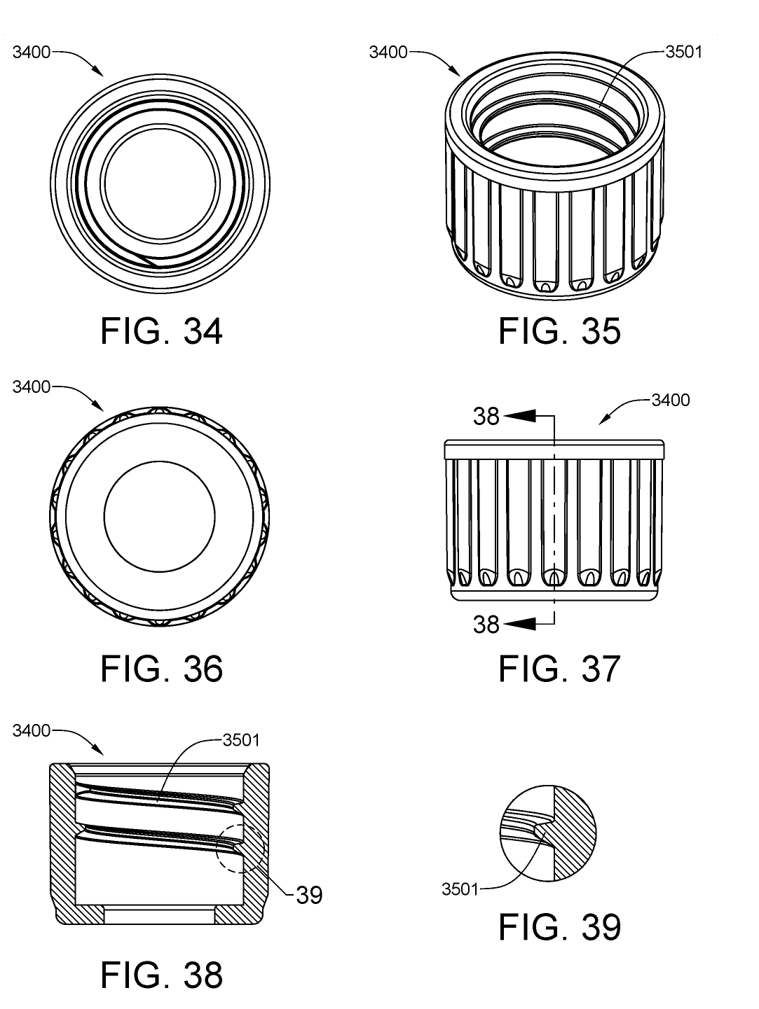

Referring now to FIGS. 34, 35, 36, 37, 38 and 39, a screw-on cap 3400 is used to install a conduction foil and polyethylene seal (see item 4601 of FIG. 46) on the opening 605 at the bottom end of the cylindrical well 3301 of the second embodiment top cover 3300. The screw-on cap 3400 is equipped with an internal thread 3501 that engages the external thread 3302 at the bottom of the well 3301. The screw-on cap 3400 presses a die-cut foil and polyethylene seal against the bottom edge of the opening 3303 at the bottom end of the well 3301. Once so installed, the bottom end of the well is subjected to a conduction process which fuses the polyethylene component of the membrane to the bottom end of the opening 3303. Installation of the membrane (see item 4501 of FIG. 45) on the opening 605 at the bottom of the well 604 of the first embodiment top cover 600 requires special equipment that applies a membrane sheet to the opening under pressure, supplies conduction heat to fuse the membrane in place, and trims the flash around the fused membrane.

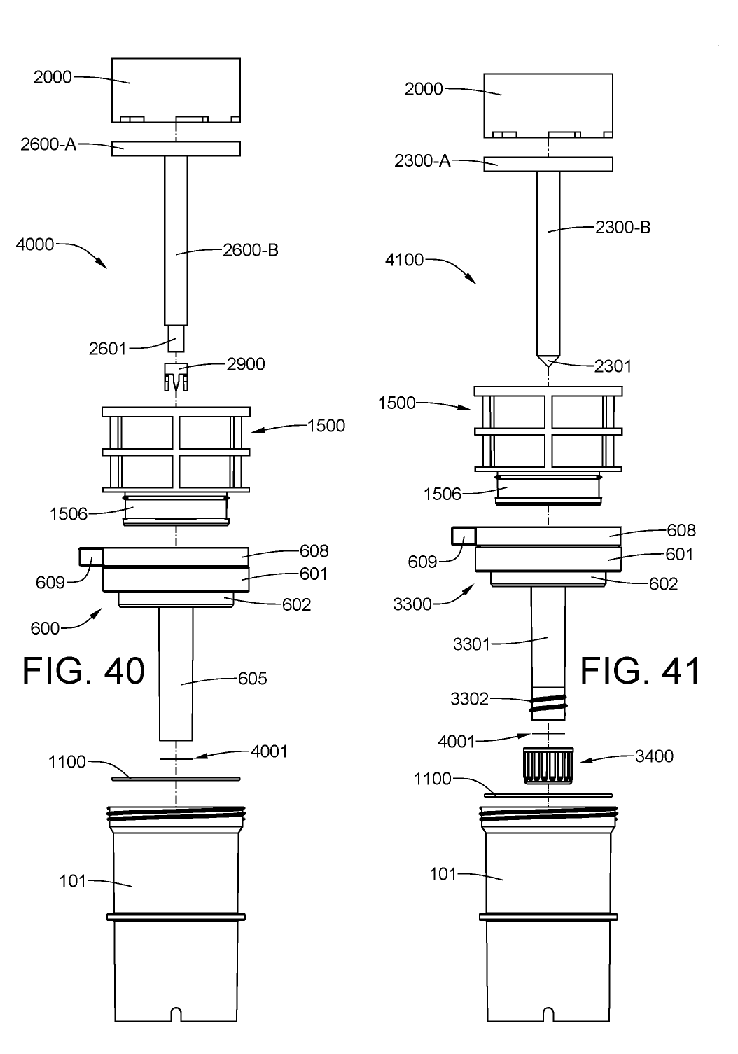

Referring now to FIG. 40, this exploded view of a first embodiment air freshener cartridge 4000 shows the incorporation of second embodiment wick pole component 2600-B and a first embodiment top cover 600. The membrane 4001 that will cover the lower opening 607 of the top cover 600 is visible in this view.

Referring now to FIG. 41, this exploded view of a second embodiment air freshener cartridge 4100 shows the incorporation of first embodiment wick pole component 2300-B and a second embodiment top cover 3300.

It should be understood that two other embodiments of the air freshener cartridge are possible. A third embodiment has a first embodiment wick pole component 2300-B and a first embodiment top cover 600. A fourth embodiment has a second embodiment wick pole component 2600-B and a second embodiment top cover 3300. It should be further understood that once assembled, all four embodiments have an identical external appearance.

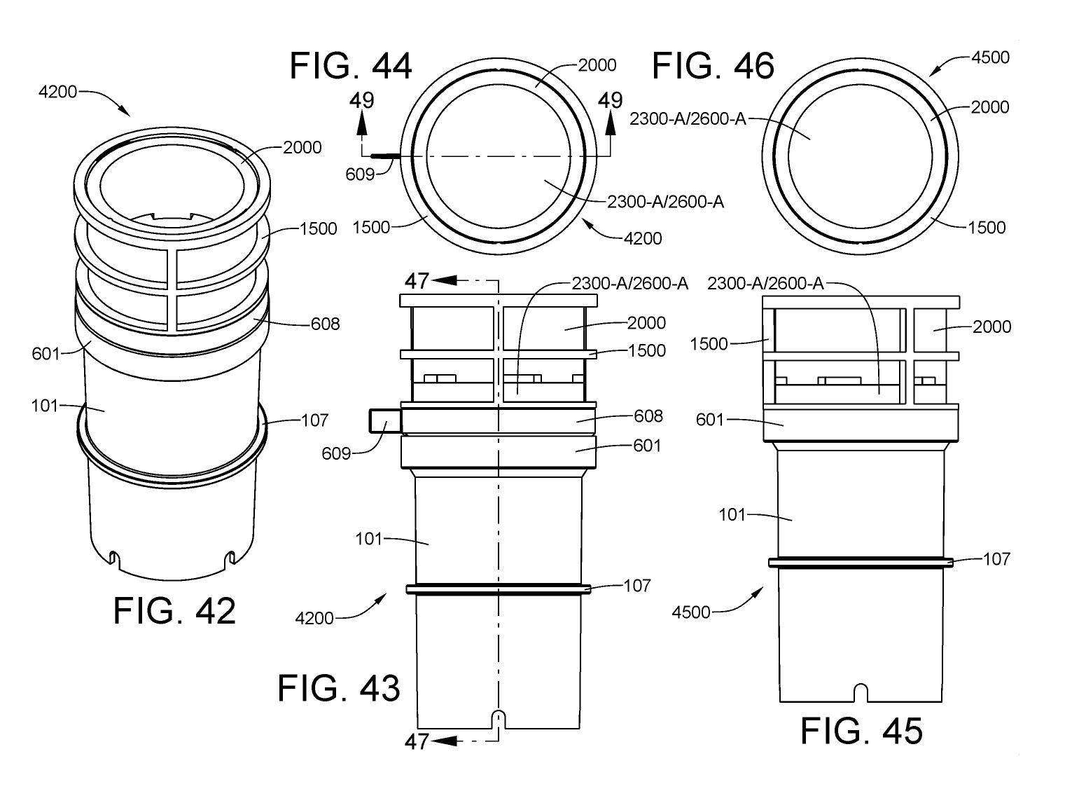

Referring now to FIGS. 42, 43 and 44, a fully assembled air freshener cartridge assembly 4200 in the shipping and storage configuration has the tear-away strip 608 still in place and the membrane 4001 covering the lower opening 607 of the liquid fragrance supply canister top cover 600 unpunctured. It should be understood that these exterior view of the air freshener cartridge assembly 4200 could include either type of wick pole component 2300-B or 2600-B and either type of top cover 600 or 3300, as those components are simply not visible in an exterior view, such as this. Thus, if the second embodiment supply canister top cover 3300 is used in the cartridge assembly 4200, the membrane 4001 covering the lower opening of that top cover is also unpunctured.

Referring now to FIGS. 45 and 46, the fully assembled air freshener cartridge assembly 4200 of FIGS. 42, 43 and 44 has been converted to an in-use configuration by removing the tear-away strip 608 and shoving the wick holder 1500 and tripartite wick downwardly so that the lower end of the wick pole component 2300-B or 2600-B pierces the membrane 4001 covering the lower opening 607 of the liquid fragrance supply canister top cover (600 or 3300) and inserts the lower end of the wick pole component 2300-B or 2600-B into the liquid fragrance supply.

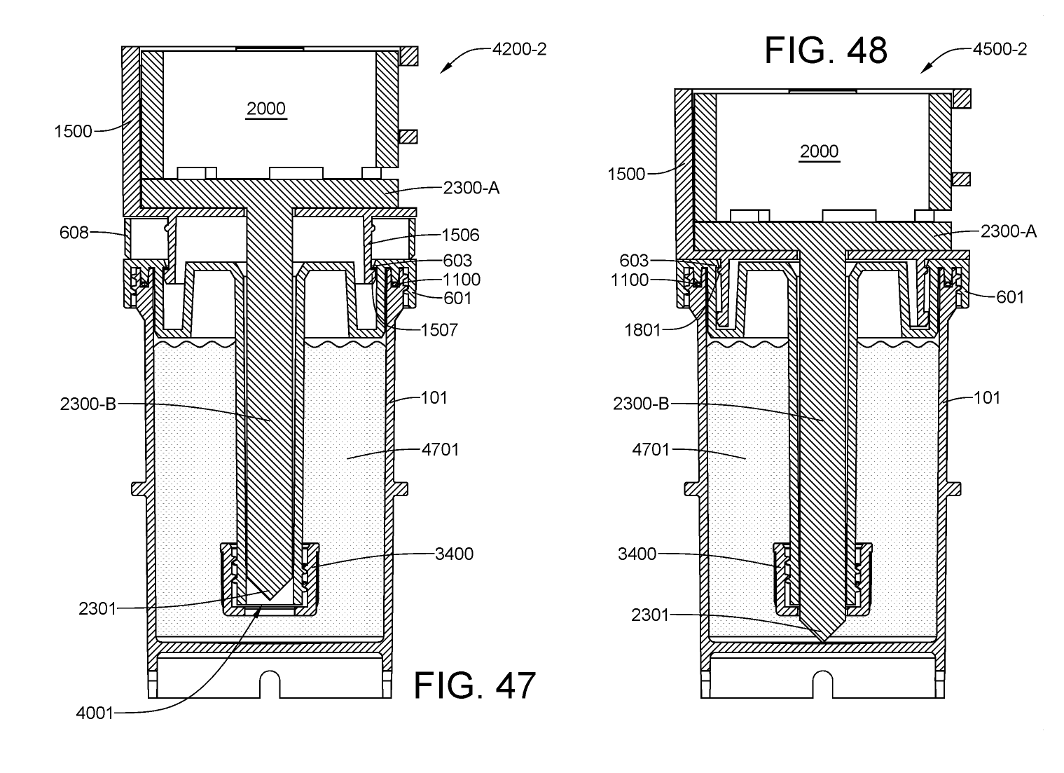

Referring now to FIG. 47, this cross-sectional view of the fully-assembled air freshener cartridge assembly 4200-2 (second embodiment cartridge) is seen in its shipping and storage configuration, with the tear-away strip 608 still in place and the membrane 4001 covering the lower opening 607 of the liquid fragrance supply canister top cover 600 unpunctured so that the liquid fragrance 4701 remains sealed in the fragrance supply cannister. It will be noted that this is an assembled version of the second embodiment air freshener cartridge assembly 4100 shown as an exploded view in FIG. 41.

Referring now to FIG. 48, this cross-sectional view of the fully-assembled air freshener cartridge assembly 4200-2 of FIG. 47 shows the assembly after it has been converted to an in-use configuration by removing the tear-away strip 608 and shoving the wick holder 1500 and tripartite wick downwardly so that the lower end of the wick pole component 2300-B pierces the membrane 4001 that covers the lower opening 607 of the liquid fragrance supply canister top cover 3300 and inserts the lower end of the wick pole component 2300-B into the liquid fragrance 4701. In this drawing, the in-use configuration of the 4200-2 cartridge assembly has been renumbered as 4500-2.

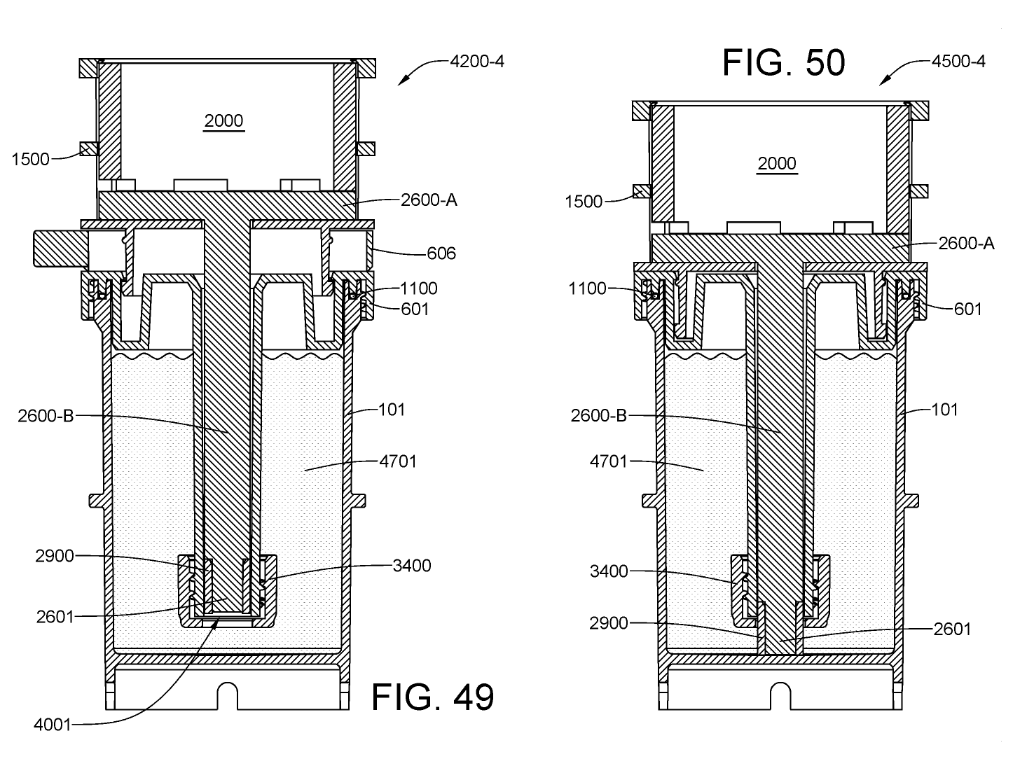

Referring now to FIG. 49, this cross-sectional view of the fully-assembled air freshener cartridge assembly 4200-4 (fourth embodiment cartridge) is seen in its shipping and storage configuration, with the tear-away strip 608 still in place and the membrane 4001 covering the lower opening 607 of the liquid fragrance supply canister top cover 600 unpunctured.

Referring now to FIG. 50, this cross-sectional view of the fully-assembled air freshener cartridge assembly 4200-4 of FIG. 49 shows the assembly after it has been converted to an in-use configuration by removing the tear-away strip 608 and shoving the wick holder 1500 and tripartite wick downwardly so that the lower end of the wick pole component 2600-B pierces the membrane 4601 on the opening 607 of the liquid fragrance supply canister top cover 3300 and inserts the lower end of the wick pole component 2600-B into the liquid fragrance 4701. In this drawing, the in-use configuration of the 4200-4 cartridge assembly has been renumbered as 4500-4.

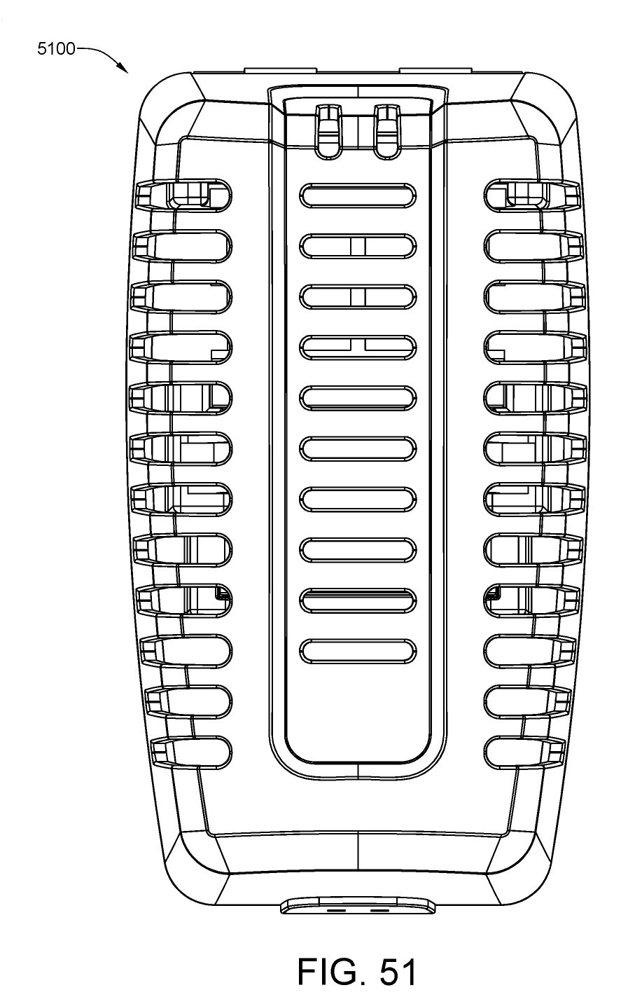

Referring now to FIG. 51, a passive air freshener dispenser 5100, of the type that can receive the air freshener cartridge of the present invention, is shown.

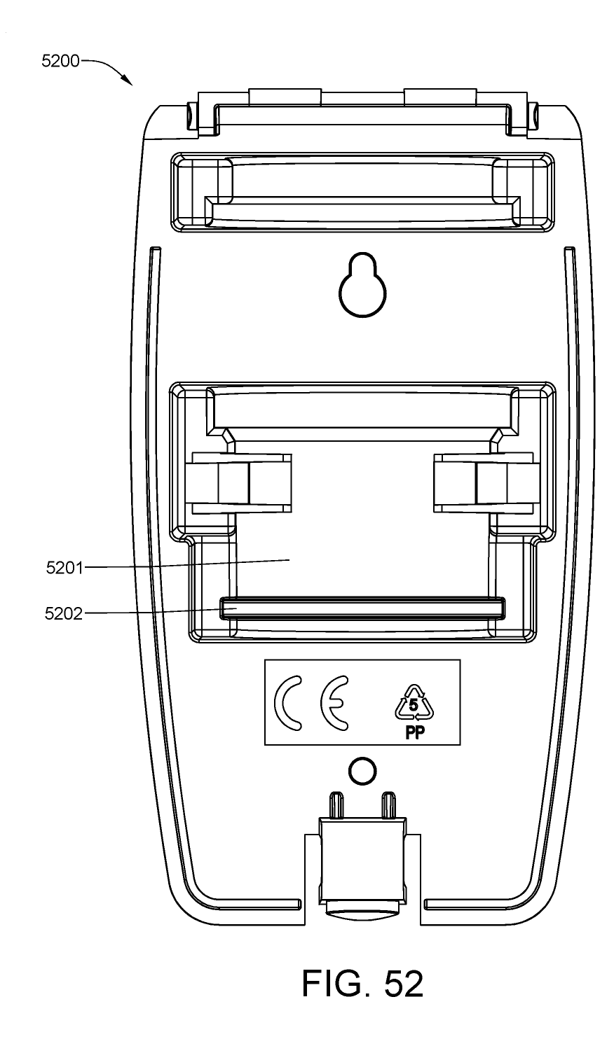

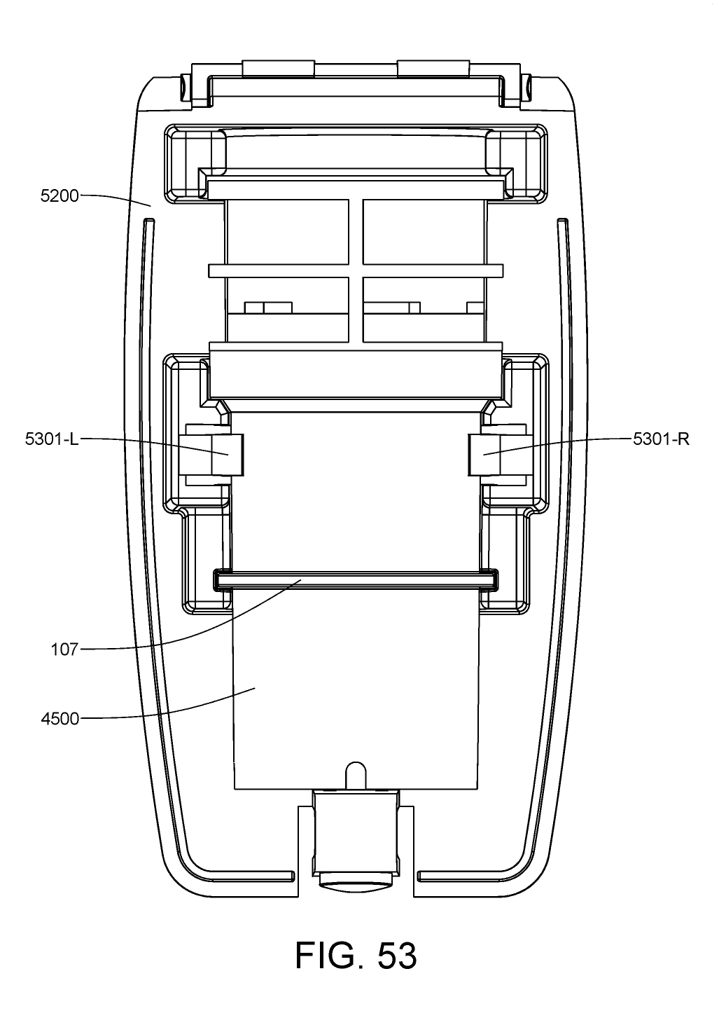

Referring now to FIG. 52, the front cover of the passive air freshener 5100 of FIG. 51 has been removed, thereby exposing the back plate 5200. It will be noted that the cartridge socket 5201 has a groove 5202, into which the lock-out flange 107 of the reservoir 100 fits.

Referring now to FIG. 53, an activated air freshener cartridge 4500 has been installed on the back plate 5200. Spring-loaded arms 5301-L and 5301-R snap over the cylindrical wall 101 of the activated air freshener cartridge 4500.

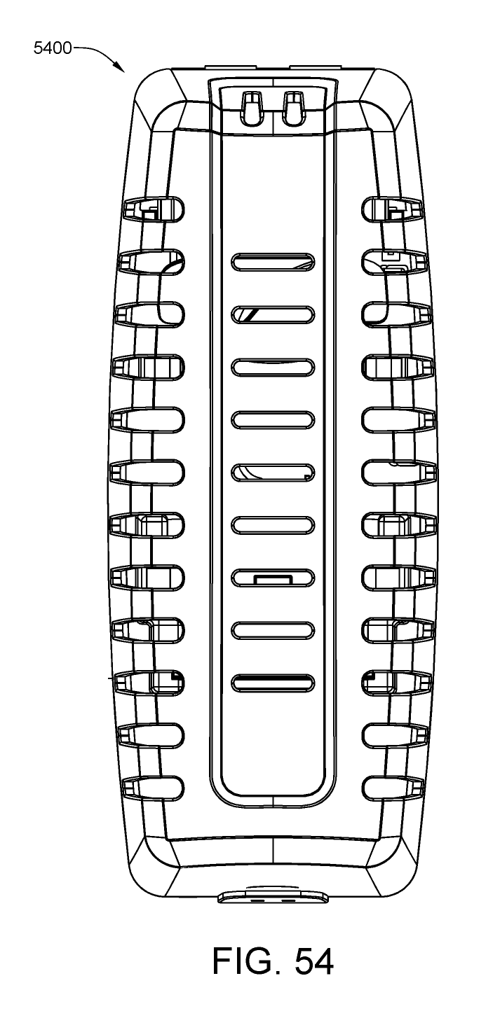

Referring now to FIG. 54, a fan-equipped air freshener dispenser 5400, of the type that can receive the air freshener cartridge of the present invention, is shown.

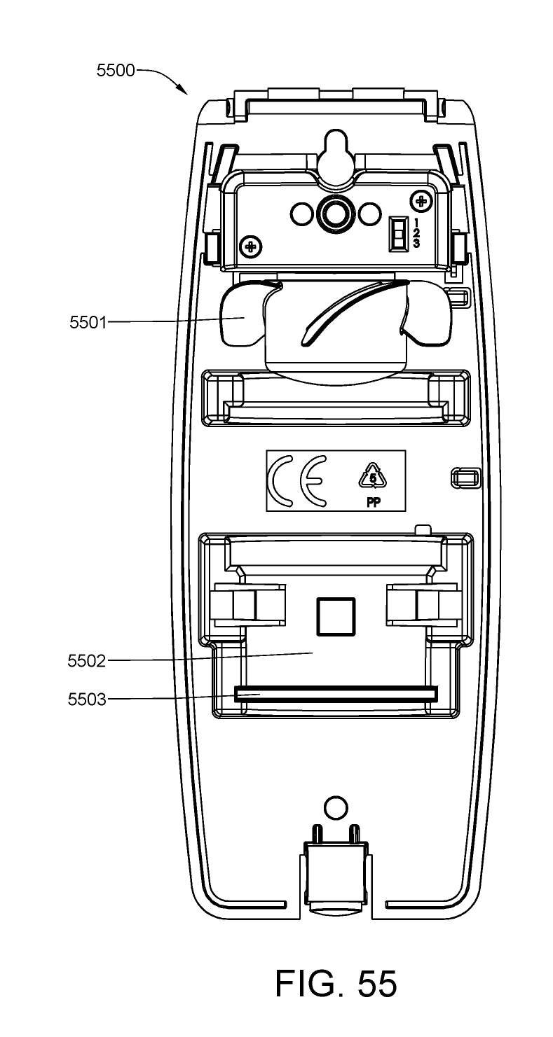

Referring now to FIG. 55, the front cover of the fan-equipped air freshener dispenser 5400 of FIG. 54 has been removed, thereby exposing the back plate 5500. The fan 5501, which is now visible, operates either continuously or intermittently, depending on the circuitry of the dispenser 5400. It will be noted that the cartridge socket 5502 has a groove 5503, into which the lock-out flange 107 of the reservoir 100 fits.

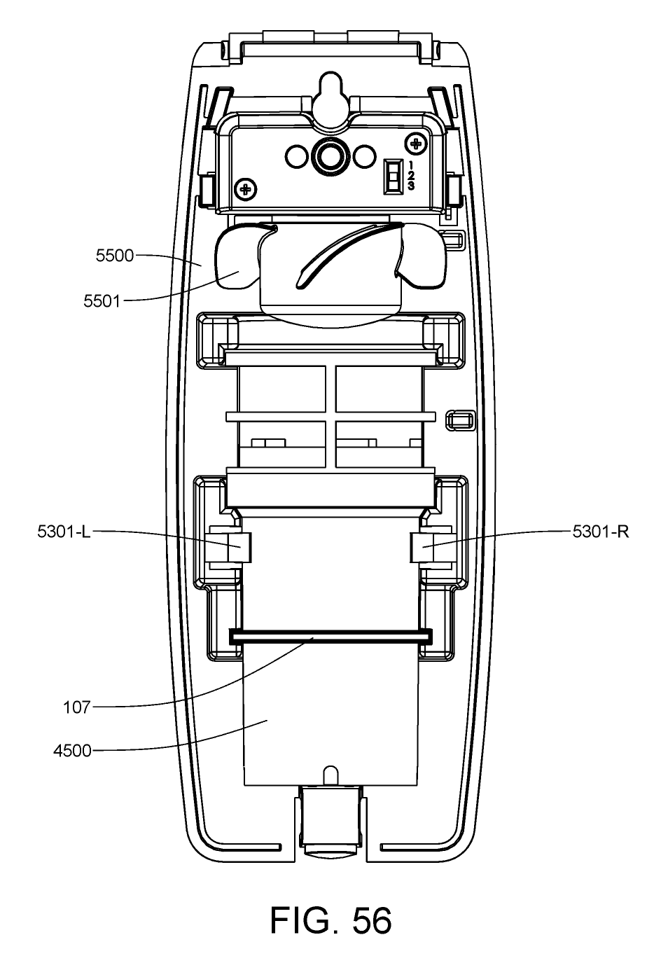

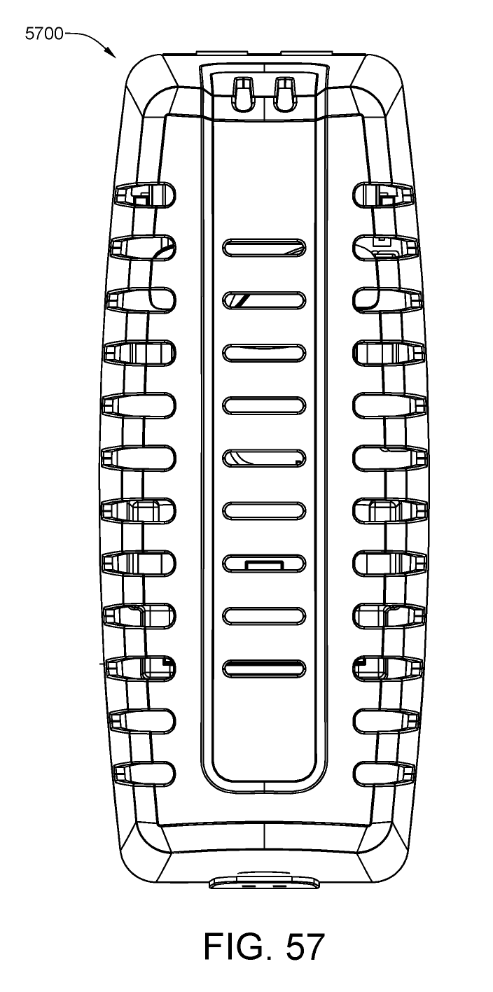

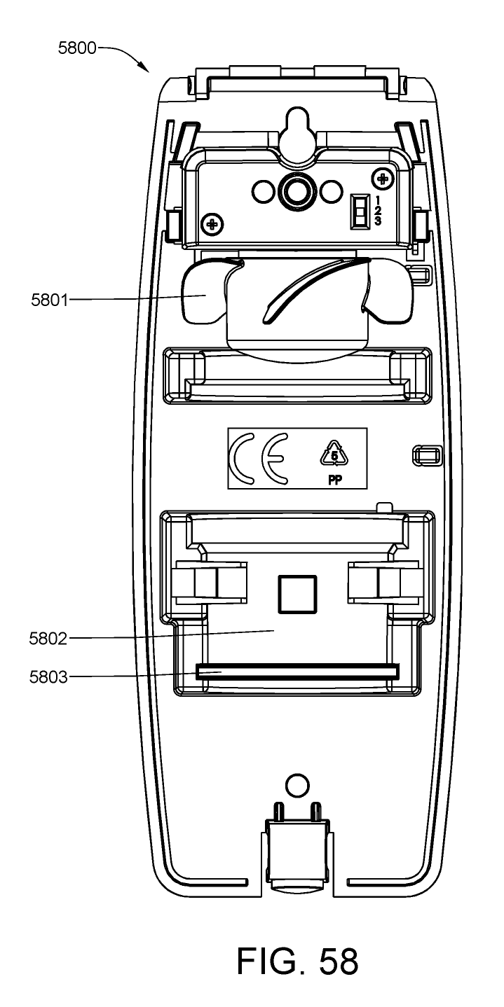

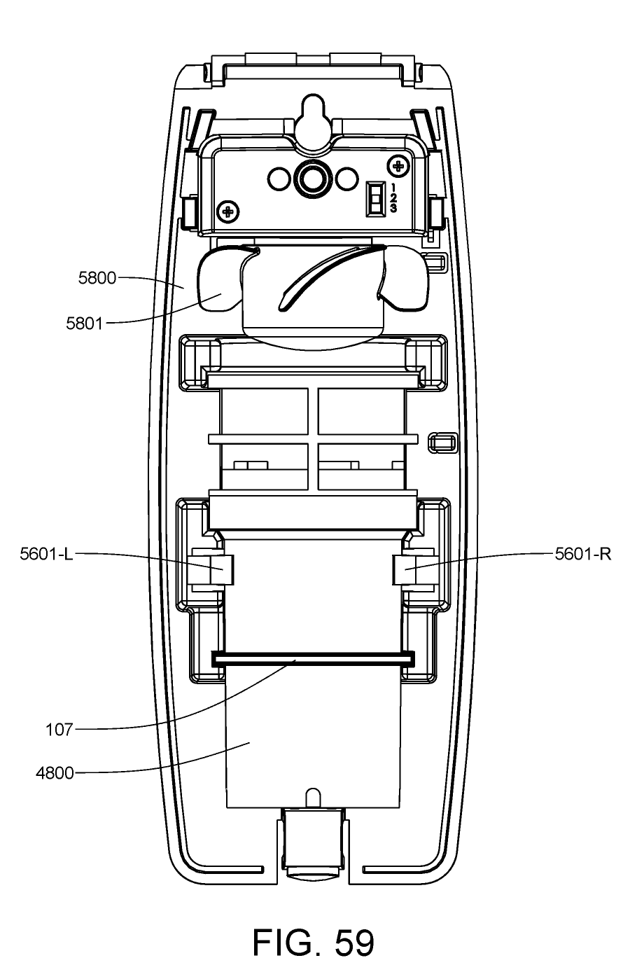

Referring now to FIG. 56, an activated air freshener cartridge 4500 has been installed on the back plate 5500. Spring-loaded arms 5601-L and 5601-R snap over the cylindrical wall 101 of the activated air freshener cartridge 4500.

Although only a single embodiment of the air freshener cartridge has been heretofore described, it will be obvious to those having ordinary skill in the art that changes and modifications may be made thereto without departing from the scope and the spirit of the invention as hereinafter claimed.

What is claimed is:

1. A spill-proof, passive air freshener refill cartridge comprising:
a fragrance supply canister having an reservoir at least partially filled with liquid fragrance and a top cover for the reservoir, said top cover having a downward projecting well, the well having a bottom opening which is below a top level of the liquid fragrance, said bottom opening being sealed closed with a puncturable membrane; and
a wick having both a pole component which can be shoved in a downwardly direction within the well so as to puncture the membrane and immerse a lower portion of the pole component within the liquid fragrance in order to activate the refill cartridge, and at least one component that is exposed to the atmosphere and from which said liquid fragrance can evaporate into the atmosphere, said exposed component being in contact with the pole component so that liquid fragrance from the pole component is transferred via capillary action to the exposed component.

2. The spill-proof, passive air freshener refill cartridge of claim 1, wherein said liquid fragrance canister incorporates a tear-away circumferential strip which, when removed, enables the wick to be pushed in a downward direction.

3. The spill-proof, passive air freshener refill cartridge of claim 1, wherein the wick comprises:
the pole component;
a laminar component, which is perpendicular to the axis of the pole component and unitary therewith; and
a wall component that is coaxial with the pole component and in contact with the laminar component.

4. The spill-proof, passive air freshener refill cartridge of claim 3, wherein the laminar component is disc shaped and the wall component is cylindrically shaped.

5. The spill-proof, passive air freshener refill cartridge of claim 4, wherein a lower edge of the cylindrical wall component is castellated.

6. The spill-proof, passive air freshener refill cartridge of claim 1, wherein a bottom end of the pole component is equipped with a rigid sharpened attachment.

7. The spill-proof, passive air freshener refill cartridge of claim 1, wherein a bottom end of the pole component is conical and terminates in a point.

8. A spill-proof, passive air freshener refill cartridge comprising:
a fragrance supply canister having a lower reservoir that is capped with a top cover which incorporates a downwardly-projecting central well, the bottom of which is below a level of liquid fragrance contained within the canister, and sealed closed with a puncturable membrane;
a wick having both a pole component which can be pushed in a downwardly direction within the well so as to puncture the membrane and immerse a lower portion of the pole component within the liquid fragrance in order to activate the refill cartridge, and a component exposed to the atmosphere, said exposed component being in contact with the pole component so that liquid fragrance from the pole component is transferred via capillary action to the exposed component from which said liquid fragrance can evaporate into the atmosphere at a controlled rate; and
a wick container that surrounds the exposed wick component, said wick container having a wick cage structure that is unitary with a base having a central aperture through which the wick pole component projects into the well.

9. The spill-proof, passive air freshener refill cartridge of claim 8, wherein said liquid fragrance canister incorporates a tear-away circumferential strip which, when removed, enables the wick to be pushed in a downward direction.

10. The spill-proof, passive air freshener refill cartridge of claim 8, wherein the wick comprises:
the pole component;
a laminar component, which is perpendicular to the axis of the pole component and unitary therewith; and
a wall component that is perpendicular to the laminar component and in contact therewith.

11. The spill-proof, passive air freshener refill cartridge of claim 10, wherein the laminar component is disc shaped and the wall component is cylindrically shaped, and wherein both the disc-shaped component and the cylindrically-shaped component are enclosed in a wick cage that clips into the top cover in one of two positions, the first being a shipping and storage position, and the second being an in-use position with a bottom portion of the pole component immersed in liquid fragrance.

12. The spill-proof, passive air freshener refill cartridge of claim 11, wherein a lower edge of the cylindrical wall component is castellated.

13. The spill-proof, passive air freshener refill cartridge of claim 8, wherein a bottom end of the pole component is equipped with a rigid sharpened attachment.

14. The spill-proof, passive air freshener refill cartridge of claim 8, wherein a bottom end of the pole component is conical and terminates in a point.

15. A spill-proof, passive air freshener refill cartridge comprising:
a fragrance supply canister having top cover with a downward projecting well, the bottom of which is below the level of liquid fragrance contained within the canister, and sealed closed with a puncturable membrane;
a tripartite wick having a cylindrical wall component, a lower surface of which is in contact with a laminar disc component, which, in turn, is unitary with a downwardly projecting pole component that is equipped with at least one sharpened feature, said tripartite wick being downwardly movable so that the sharpened feature of the pole punctures the membrane and a lower portion of the pole component becomes immersed in the liquid fragrance in order to activate the refill cartridge, thereby enabling liquid fragrance to climb the pole component via capillary action and enter both the laminar disk component and the cylindrical wall component which are both at least partially exposed to the atmosphere, and from which liquid fragrance can evaporate at a controlled rate; and
a wick holder that surrounds the exposed wick components, said wick container having a wick cage structure that is unitary with a base having a central aperture through which the wick pole component projects into the well.

16. The spill-proof, passive air freshener refill cartridge of claim 15, wherein the top cover of said liquid fragrance canister incorporates a tear-away circumferential strip which, when removed, enables the wick to be pushed in a downward direction.

17. The spill-proof, passive air freshener refill cartridge of claim 15, wherein the laminar component is disc shaped and the wall component is cylindrically shaped, and wherein both the disc-shaped component and the cylindrically-shaped component are enclosed in a wick cage that clips into the top cover in one of two positions, the first being a shipping and storage position, and the second being an in-use position with a bottom portion of the pole component immersed in liquid fragrance.

18. The spill-proof, passive air freshener refill cartridge of claim 15, wherein a bottom end of the pole component is equipped with a rigid sharpened attachment.

19. The spill-proof, passive air freshener refill cartridge of claim 15, wherein a bottom end of the pole component is conical and terminates in a point.

20. The spill-proof, passive air freshener refill cartridge of claim 15, wherein the pole component has a diameter that is slightly larger than the opening at the bottom of the well so that the pole component is compressed by the opening so that spillage of liquid fragrance cannot occur if the cartridge were to fall over on is side.
